SEQUENCE LISTING (26 sequences)

SEQ ID NO: 1 - DNA, 22 bp, synthetic construct
  source 1..22: mol_type = other DNA
  misc_feature 1..22: note = MMP1 forward primer
cccaaaagcgtgtgacagtaag

SEQ ID NO: 2 - DNA, 21 bp, synthetic construct
  source 1..21: mol_type = other DNA
  misc_feature 1..21: note = MMP1 reverse primer
cttccgggtagaagggatttg

SEQ ID NO: 3 - DNA, 23 bp, synthetic construct
  source 1..23: mol_type = other DNA
  misc_feature 1..23: note = MMP2 forward primer
gcacccatttacacctacaccaa

SEQ ID NO: 4 - DNA, 21 bp, synthetic construct
  source 1..21: mol_type = other DNA
  misc_feature 1..21: note = MMP2 reverse primer
agagctcctgaatgcccttga

SEQ ID NO: 5 - DNA, 20 bp, synthetic construct
  source 1..20: mol_type = other DNA
  misc_feature 1..20: note = MMP3 forward primer
cgtgaggaaaatcgatgcag

SEQ ID NO: 6 - DNA, 23 bp, synthetic construct
  source 1..23: mol_type = other DNA
  misc_feature 1..23: note = MMP3 reverse primer
cttcagctatttgcttgggaaag

SEQ ID NO: 7 - DNA, 20 bp, synthetic construct
  source 1..20: mol_type = other DNA
  misc_feature 1..20: note = MMP9 forward primer
agtccacccttgtgctcttc

SEQ ID NO: 8 - DNA, 19 bp, synthetic construct
  source 1..19: mol_type = other DNA
  misc_feature 1..19: note = MMP9 reverse primer
tttcgactctccacgcatc

SEQ ID NO: 9 - DNA, 19 bp, synthetic construct
  source 1..19: mol_type = other DNA
  misc_feature 1..19: note = MT1-MMP1 forward primer
tcagggcagtggatagcga

SEQ ID NO: 10 - DNA, 21 bp, synthetic construct
  source 1..21: mol_type = other DNA
  misc_feature 1..21: note = MT1-MMP1 reverse primer
gccggttctaccttcagcttc

SEQ ID NO: 11 - DNA, 21 bp, synthetic construct
  source 1..21: mol_type = other DNA
  misc_feature 1..21: note = ITGA1 forward primer
cagccccacatttcaagtcgt

SEQ ID NO: 12 - DNA, 21 bp, synthetic construct
  source 1..21: mol_type = other DNA
  misc_feature 1..21: note = ITGA1 reverse primer
acctgtgtctgtttaggacca

SEQ ID NO: 13 - DNA, 21 bp, synthetic construct
  source 1..21: mol_type = other DNA
  misc_feature 1..21: note = ITGA2 forward primer
gcaactggttactggttggtt

SEQ ID NO: 14 - DNA, 23 bp, synthetic construct
  source 1..23: mol_type = other DNA
  misc_feature 1..23: note = ITGA2 reverse primer
gaggctcatgttggttttcatct

SEQ ID NO: 15 - DNA, 22 bp, synthetic construct
  source 1..22: mol_type = other DNA
  misc_feature 1..22: note = ITGA10 forward primer
cttcagttctgggatatgtgcc

SEQ ID NO: 16 - DNA, 21 bp, synthetic construct
  source 1..21: mol_type = other DNA
  misc_feature 1..21: note = ITGA10 reverse primer
ccagtcttcgtaggaaggtct

SEQ ID NO: 17 - DNA, 21 bp, synthetic construct
  source 1..21: mol_type = other DNA
  misc_feature 1..21: note = ITGA11 forward primer
tcacggacaccttcaacatgg

SEQ ID NO: 18 - DNA, 21 bp, synthetic construct
  source 1..21: mol_type = other DNA
  misc_feature 1..21: note = ITGA11 reverse primer
ccagccacttattgccactga

SEQ ID NO: 19 - DNA, 21 bp, synthetic construct
  source 1..21: mol_type = other DNA
  misc_feature 1..21: note = GAPDH forward primer
tctctgctcctcctgttcgac

SEQ ID NO: 20 - DNA, 22 bp, synthetic construct
  source 1..22: mol_type = other DNA
  misc_feature 1..22: note = GAPDH reverse primer
gttgactccgaccttcaccttc

SEQ ID NO: 21 - DNA, 20 bp, synthetic construct
  source 1..20: mol_type = other DNA
  misc_feature 1..20: note = MMP13 forward primer
cttcccaaccgtattgatgc

SEQ ID NO: 22 - DNA, 23 bp, synthetic construct
  source 1..23: mol_type = other DNA
  misc_feature 1..23: note = MMP13 reverse primer
acttcttttggaagacccagttc

SEQ ID NO: 23 - DNA, 20 bp, synthetic construct
  source 1..20: mol_type = other DNA
  misc_feature 1..20: note = HS OCT4 forward primer
ctcctggagggccaggaatc

SEQ ID NO: 24 - DNA, 20 bp, synthetic construct
  source 1..20: mol_type = other DNA
  misc_feature 1..20: note = HS OCT4 reverse primer
ccacatcggcctgtgtatat

SEQ ID NO: 25 - DNA, 20 bp, synthetic construct
  source 1..20: mol_type = other DNA
  misc_feature 1..20: note = HS NANOG forward primer
caaaggcaaacaacccactt

SEQ ID NO: 26 - DNA, 20 bp, synthetic construct
  source 1..20: mol_type = other DNA
  misc_feature 1..20: note = HS NANOG reverse primer
tctgctggaggctgaggtat

(19) United States
(12) Patent Application Publication
(10) Pub. No.: US 2025/0339584 A1
(43) Pub. Date: Nov. 6, 2025

(54) COMPOSITION FOR CARTILAGE REPAIR AND METHOD FOR MANUFACTURING SAME

(71) Applicant: OSAKA UNIVERSITY, Osaka (JP)

(72) Inventors: Ken NAKATA, Osaka (JP); Takashi KANAMOTO, Osaka (JP); Shunya OTANI, Osaka (JP)

(73) Assignee: OSAKA UNIVERSITY, Osaka (JP)

(21) Appl. No.: 18/998,131

(22) PCT Filed: Jul. 24, 2023
(86) PCT No.: PCT/JP2023/026944

§ 371 Date: Jan. 24, 2025

(30) Foreign Application Priority Data
Jul. 26, 2022 (JP) 2022-118591

(51) Int. Cl. A61L 27/36

(52) U.S. Cl. CPC A61L 27/3604; A61L 2430/06

(57) ABSTRACT

Provided are a novel composition for cartilage repair including a cell having a feature suitable for cartilage repair, and a production method therefor. The inventors of the present invention have paid attention to an umbilical cord tissue-derived cell, and have performed the three-dimensional culture of the umbilical cord tissue-derived cell. The inventors have found that the umbilical cord tissue-derived cell for forming a cultured product obtained by the three-dimensional culture has the following features: its cell proliferation potency and gene expression level are different from those of a cell for forming a cultured product obtained by the three-dimensional culture of cells derived from any other tissue. The umbilical cord tissue-derived cell of the present invention for forming the cultured product obtained by the three-dimensional culture has a feature suitable for cartilage repair, and hence can provide a composition effective in the cartilage repair.

### TECHNICAL FIELD

The present invention relates to a composition for cartilage repair including, as an active ingredient, a three-dimensionally cultured product formed of an umbilical cord tissue-derived cell, and a production method for a composition for cartilage repair.

The present application claims priority from Japanese Patent Application No. 2022-118591, which is incorporated herein by reference.

### BACKGROUND ART

In general, a stable treatment outcome is obtained for a musculoskeletal disease, such as a bone fracture or musculoskeletal damage, by selecting gypsum treatment, rehabilitation or surgical treatment, or the like in accordance with an individual case. Meanwhile, there exists such a disease as described below: even when treatment considered to be best for the disease is performed, there is a high risk of the remaining of dysfunction, or the recurrence or worsening of the disease thereafter. A disease of a cartilage typified by a knee meniscus or an articular cartilage is a typical example of such disease, and a cause therefor is, for example, the fact that the cartilage is poor in tissue healing power.

The supply of a cell, an extracellular matrix (scaffold), or a growth factor serves as a central approach to promoting tissue healing. In the field of the cell therapy of a joint tissue, there are many reports in each of which a mesenchymal stem cell (MSC) derived from a synovial tissue is used. The collection of a mesenchymal tissue typified by a synovium requires several times of surgery, and hence a load on a patient is large.

Umbilical cord blood and an umbilical cord tissue can be collected as by-products at the time of a delivery, and hence have an advantage in that the blood and the tissue can be collected more easily and more noninvasively than a general mesenchymal tissue can be. With regard to cartilage regeneration with a mesenchymal stem cell derived from the umbilical cord blood, in Non Patent Literature 1, there is a report of cartilage regeneration in a defective site of the articular cartilage of a patient of arthrosis deformans by the transplantation of a pharmaceutical formed of a composite material of a human umbilical cord blood-derived mesenchymal stem cell and a hyaluronic acid hydrogel. In Patent Literature 1, there is a disclosure of a composition for treating articular cartilage damage including an umbilical cord blood-derived mesenchymal stem cell. However, only a small amount of mesenchymal stem cells are present in the umbilical cord blood, though many hematopoietic stem cells are present therein. Accordingly, it is difficult to stably obtain a homogeneous cell population of the mesenchymal stem cells.

Meanwhile, many mesenchymal stem cells are present in the umbilical cord tissue. There has been performed clinical research exploiting the anti-inflammatory or immunosuppressive ability of a mesenchymal stem cell derived from the umbilical cord tissue. Meanwhile, the number of reports on regeneration treatment in which a tissue-repairing ability is expected is small as compared to a mesenchymal stem cell derived from any other tissue (myeloid, fat, or joint tissue). With regard to cartilage regeneration with a mesenchymal stem cell derived from the umbilical cord tissue, in Patent Literature 2, there are disclosures of cartilage regeneration in a damaged site of the cartilage of a rabbit knee joint by the transplantation of a chondrocyte therapeutic agent containing a human umbilical cord-derived stem cell, and the three-dimensional culture of the human umbilical cord-derived stem cell, a hyaluronic acid derivative, and collagen. In Non Patent Literature 2, there is a report of cartilage repair in a damaged site of the cartilage of a miniature pig knee joint by the transplantation of the mixture of a human umbilical cord-derived mesenchymal stem cell and hyaluronic acid. However, in each of Patent Literature 2 and Non Patent Literature 2, no reference is made to cartilage repair with a three-dimensionally cultured product formed of an umbilical cord tissue-derived cell, the comparison of the expression levels of a matrix metalloprotease (MMP) gene and an integrin a subunit (ITGA) gene with a cell derived from any other tissue, and the expression of each of an Oct4 gene and a Nanog gene.

### CITATION LIST

### Non Patent Literature

[NPL 1] Stem Cells Transl Med. 2017 February; 6 (2): 613-621.
[NPL 2] Tzu Chi Med J. 2019 January-Mar; 31 (1): 11-19.

### PATENT LITERATURE

[PTL 1] JP 2012-107026 A
[PTL 2] JP 2014-520844 A

### SUMMARY OF INVENTION

### Technical Problem

In a cartilage tissue, chondrocytes are three-dimensionally present through an extracellular matrix. The three-dimensional culture of a cell having a differentiation ability enables its application to regenerative medicine in a state closer to the inside of a living body. An object of the present invention is to provide a novel composition for cartilage repair including a cell having a feature suitable for cartilage repair.

### Solution to Problem

To solve the above-mentioned problems, the inventors of the present invention have paid attention to an umbilical cord tissue-derived cell. The inventors have made extensive investigations and performed the three-dimensional culture of the umbilical cord tissue-derived cell. The inventors have found that the umbilical cord tissue-derived cell for forming a cultured product obtained by the three-dimensional culture has the following features: its cell proliferation potency and gene expression level are different from those of a cell for forming a cultured product obtained by the three-dimensional culture of cells derived from any other tissue. Thus, the inventors have completed the present invention.

That is, the present invention includes the following.
1. A composition for cartilage repair, including, as an active ingredient, a three-dimensionally cultured product formed of an umbilical cord tissue-derived cell.
2. The composition for cartilage repair according to the above-mentioned item 1, wherein an expression level of a matrix metalloprotease gene in the umbilical cord tissue-derived cell for forming the three-dimensionally cultured product is lower than an expression level of the gene in a synovial tissue-derived cell or a meniscus-derived cell.
3. The composition for cartilage repair according to the above-mentioned item 1 or 2, wherein the matrix metalloprotease gene is one or a plurality of kinds of genes selected from the group consisting of: MMP1; MMP2; MMP3; MMP9; MMP13; and MT1-MMP.
4. The composition for cartilage repair according to any one of the above-mentioned items 1 to 3,
wherein expression levels of one or two kinds of ITGA1 and ITGA2 genes in the umbilical cord tissue-derived cell for forming the three-dimensionally cultured product are higher than an expression level of each of the genes in a synovial tissue-derived cell or a meniscus-derived cell, and/or
wherein expression levels of one or two kinds of ITGA10 and ITGA11 genes in the umbilical cord tissue-derived cell for forming the three-dimensionally cultured product are lower than an expression level of each of the genes in the synovial tissue-derived cell or the meniscus-derived cell.
5. The composition for cartilage repair according to any one of the above-mentioned items 1 to 4, wherein the three-dimensionally cultured product contains an umbilical cord tissue-derived cell cultured in the presence of a scaffold substrate.
6. The composition for cartilage repair according to any one of the above-mentioned items 1 to 5, wherein expression levels of one or two kinds of Oct4 and Nanog genes in the umbilical cord tissue-derived cell are higher than an expression level of each of the genes in an umbilical cord tissue-derived cell cultured in the absence of the scaffold substrate.
7. The composition for cartilage repair according to any one of the above-mentioned items 1 to 6, wherein a cartilage in the composition for cartilage repair is one or a plurality of kinds of cartilages selected from the group consisting of: a meniscus; an articular cartilage; an intervertebral disc; a costal cartilage; a tracheal cartilage; a bronchial cartilage; a nasal cartilage; a thyroid cartilage; a pubic symphysis; an articular disc; an epiphyseal plate; an auricular cartilage; a meatal cartilage; an epiglottic cartilage; a laryngeal cartilage; and an articular lip.
8. The composition for cartilage repair according to any one of the above-mentioned items 1 to 7, wherein the umbilical cord tissue-derived cell includes an undifferentiated cell.
9. A production method for a composition for cartilage repair, including a step of three-dimensionally culturing an umbilical cord tissue-derived cell.
10. The production method according to the above-mentioned item 9, wherein the step of three-dimensionally culturing the umbilical cord tissue-derived cell includes a step of culturing the umbilical cord tissue-derived cell in the presence of a scaffold substrate.

### Advantageous Effects of Invention

The umbilical cord tissue-derived cell of the present invention for forming the cultured product obtained by the three-dimensional culture has the following property: the cell is low in expression level of the matrix protease gene (e.g., MMP1) as compared to a cell for forming a cultured product obtained by the three-dimensional culture of the synovial tissue-derived cell or the meniscus-derived cell, and/or is high in expression level of a specific integrin a subunit gene (e.g., ITGA1) as compared thereto, and/or is low in expression level of another specific integrin a subunit gene (e.g., ITGA10) as compared thereto, and/or is high in expression level of a gene (e.g., an Oct4 gene) involved in an undifferentiation property as compared to the umbilical cord tissue-derived cell cultured in the absence of the scaffold substrate. Thus, the composition effective in cartilage repair is provided.

### BRIEF DESCRIPTION OF DRAWINGS

FIG. 1 are histograms for showing the results of the expression analysis of MSC cell surface markers. In the histograms, the relative expression of each of (A) CD73, (B) CD90, (C) CD105, (D) CD34, and (E) CD45 is displayed while an isotype control is superimposed thereon. In FIG. 1A to FIG. 1C, right-leaning histograms are the datasets of (A) an anti-CD73 antibody, (B) an anti-CD90 antibody, and (C) an anti-CD105 antibody, respectively, and left-leaning histograms are control histograms each using an anti-IgG antibody. In FIG. 1D and FIG. 1E, the datasets of (D) an anti-CD34 antibody and (E) an anti-CD45 antibody each showed a histogram coinciding with the isotype control using the anti-IgG antibody. In each of the figures, an axis of ordinate indicates a fluorescence intensity, and an axis of abscissa indicates the number of cells (Example 1).

FIG. 2 are photographs for showing the results of the evaluation of a MSC differentiation ability. FIG. 2A is a photograph for showing an undifferentiated umbilical cord tissue-derived cell, FIG. 2B is a photograph for showing a cell (stained with Oil Red) that has differentiated into an adipocyte through culture in an adipocyte differentiation medium, FIG. 2C is a photograph for showing a cell (stained with Alizarin Red) that has differentiated into an osteoblast through culture in an osteoblast differentiation medium, and FIG. 2D is a photograph for showing a cell (stained with Alcian Blue) that has differentiated into a chondrocyte through culture in a chondrocyte differentiation medium (Example 1).

FIG. 3 are graphs for showing the results of comparison between the relative DNA amounts of mesenchymal stem cells derived from various tissues at each of the following time points of three-dimensional culture: 1st, 4th, 7th, 14th, and 28th days. FIG. 3A is a graph for showing the results of comparison among an umbilical cord tissue-derived mesenchymal stem cell, a synovial tissue-derived mesenchymal stem cell, and a meniscus tissue-derived mesenchymal stem cell in the three-dimensional culture, and FIG. 3B is a graph for showing the results of comparison among the umbilical cord tissue-derived mesenchymal stem cell, a fat tissue-derived mesenchymal stem cell, and a cancellous bone tissue-derived mesenchymal stem cell in the three-dimensional culture (Example 3).

FIG. 4 are graphs for showing the results of comparison between the gene expression levels of the mesenchymal stem cells derived from the various tissues on the 4th day of the three-dimensional culture. FIG. 4A, FIG. 4B, FIG. 4C, FIG. 4D, FIG. 4E, FIG. 4F, FIG. 4G, FIG. 4H, FIG. 4I, and FIG. 4J are graphs for showing the results of the measurement of MMP1, MMP2, MMP3, MMP9, MT1-MMP, MMP13, ITGA1, ITGA2, ITGA10, and ITGA11, respectively. In each of the figures, the axis of ordinate “Relative gene expression” means a relative gene expression level (Example 4).

FIG. 5 are graphs for showing the results of the gene expression analysis of plate culture and the three-dimensional culture. FIG. 5A is a graph for showing the results of the measurement of Oct4, and FIG. 5B is a graph for showing the results of the measurement of Nanog. In each of the figures, the axis of ordinate “Relative expression” means a relative gene expression level (Example 5).

### DESCRIPTION OF EMBODIMENTS

The present invention relates to a composition for cartilage repair including, as an active ingredient, a three-dimensionally cultured product formed of an umbilical cord tissue-derived cell. The present invention also relates to a production method for the composition for cartilage repair.

The term “umbilical cord tissue” as used herein refers to a tubular tissue connecting a fetus and a placenta to each other, and does not encompass umbilical cord blood in concept. Although the umbilical cord tissue of the present invention is not particularly limited as long as the umbilical cord tissue is collected from a mammal, the tissue is, for example, an umbilical cord tissue of a human, a monkey, cattle, a horse, a pig, a sheep, a dog, or a cat, and a human umbilical cord tissue is preferred.

### (Umbilical Cord Tissue-derived Cell)

The umbilical cord tissue-derived cell of the present invention is derived from a cell present in the umbilical cord tissue, and is obtained through isolation from the umbilical cord tissue. A cell population obtained from the umbilical cord tissue is a cell population including an undifferentiated cell, and the term “umbilical cord tissue-derived cell” as used herein refers to an undifferentiated cell derived from a cell present in the umbilical cord tissue unless otherwise stated. Herein, the undifferentiated cell means a cell, which has not undergone the final differentiation, and encompasses all cells in the course of their differentiation into the finally differentiated cells. The umbilical cord tissue-derived cell of the present invention encompasses all umbilical cord tissue-derived cells each having cartilage repair activity except a cell that has lost its cartilage repair activity as a result of terminal differentiation, and examples thereof include: a stem cell, a mesenchymal stem cell, an endothelial cell, and an interstitial cell derived from the umbilical cord tissue; and various progenitor cells (e.g., a cartilage progenitor cell, an osteoprogenitor cell, and a preadipocyte) in an umbilical cord interstitial tissue (umbilical cord matrix). Of those, a stem cell is preferred, and a mesenchymal stem cell is more preferred. Although an umbilical cord tissue-derived mesenchymal stem cell is not particularly limited, the cell is preferably a cell satisfying at least one of the following: the cell is positive for CD73, CD90, and/or CD105; the cell is negative for CD34 and/or CD45; and the cell has an ability to differentiate into a chondrocyte, an osteoblast, and/or an adipocyte. The cell is more preferably a cell, which is positive for the CD73, the CD90, and the CD105, is negative for the CD34 and the CD45, and has an ability to differentiate into a chondrocyte, an osteoblast, and an adipocyte. The umbilical cord tissue-derived cell of the present invention may be an autologous cell or an allogeneic cell, or may be a known cell. For example, a human umbilical cord Wharton's jelly-derived mesenchymal stem cell manufactured by Vitality, and human umbilical cord vein endothelial cells manufactured by ScienCell Research Laboratories, Inc. and PromoCell GmbH are available.

A method of isolating the umbilical cord tissue-derived cell from the umbilical cord tissue is not particularly limited, and a method known per se or a method to be developed in the future is available. Examples of the method known per se include: a method including washing the umbilical cord tissue with a buffer solution, cutting the tissue into a small piece, then culturing the small piece, and treating an adherent cell that has migrated to the outside of the tissue with a type I collagenase-containing medium after its appearance (Patent Literature 2); a method including washing the umbilical cord tissue with the buffer solution, cutting the tissue into a small piece, then treating the small piece with the type I collagenase-containing medium, culturing the resultant explant in a medium containing fetal bovine serum and an antibiotic, and obtaining a cell that has migrated to the outside of the tissue (Non Patent Literature 2); and a method including cutting a vascular tissue and a Wharton's jelly separated from the umbilical cord tissue into small pieces, and then dissociating the cell with an automatic tissue-dispersing/crushing apparatus or a sieve (JP 2013-514072 A). As one preferred aspect of the method of isolating the umbilical cord tissue-derived cell from the umbilical cord tissue, the isolation may be performed by: turning the umbilical cord tissue into a small piece with, for example, a scalpel, a mixer, and/or a homogenizer; culturing the tissue piece in a medium containing fetal bovine serum or the like; and subjecting an adherent cell that has migrated to the outside of the tissue as a result of the culture to, for example, filtration, a centrifuge, and/or pipetting. As another preferred aspect thereof, the isolation may be performed by: turning the umbilical cord tissue into a small piece with, for example, a scalpel, a mixer, and/or a homogenizer; treating the tissue piece in a liquid containing a protease such as a collagenase; and subjecting the treated piece to, for example, filtration, a centrifuge, and/or pipetting.

In the present invention, the umbilical cord tissue-derived cell isolated from the umbilical cord tissue may be passaged and maintained by monolayer culture (also referred to as “plate culture,” the same holds true for the following). In general, cell culture methods are roughly classified into adhesion culture and suspension culture, and the monolayer culture is a kind of adhesion culture. In the monolayer culture, cells adhere through use of the surface of a substrate, such as an incubator or a bead-like carrier, as a scaffold, and the cells two-dimensionally proliferate until the cells completely fill the surface, thereby forming a monolayer. The incubator for the plate culture in the present invention is, for example, an incubator subjected to cell adhesion treatment, and for example, a culture flask, a petri dish, or a multi-well plate may be used. Specifically, for example, a polystyrene dish or multi-well plate for tissue culture may be used.

The term “three-dimensional culture” as used herein refers to the following culture: a cell is cultured while being caused to interact with a surrounding environment including, for example, a surrounding cell, extracellular matrix, and/or scaffold substrate. In one aspect, the three-dimensional culture may be the culture of the cell in the presence of the scaffold substrate. The term “three-dimensionally cultured product” as used herein refers to a cultured product obtained by the three-dimensional culture of the cell. In one aspect, the three-dimensionally cultured product contains the cell cultured in the presence of the scaffold substrate and/or the scaffold substrate. The term “three-dimensionally cultured product formed of an umbilical cord tissue-derived cell” as used herein is not limited to a state in which the cultured product is formed only of the umbilical cord tissue-derived cell. The cultured product contains the umbilical cord tissue-derived cell, preferably contains the umbilical cord tissue-derived cell as a main constituent, and may contain a component except the umbilical cord tissue-derived cell, such as an extracellular matrix, a scaffold substrate, or a medium component. In the three-dimensional culture of the present invention, in addition to the incubator, the following scaffold substrate is used: a cell can be embedded in the substrate, and/or the cell can grow and/or proliferate on the surface of the substrate. For example, when a collagen sponge is used as a scaffold substrate, the cell can adhere and migrate between the pores of the porous structure of the collagen sponge, and can three-dimensionally proliferate in, and/or on the surface of, the collagen sponge. For example, when a collagen gel is used as a scaffold substrate, the cell can be embedded in the collagen gel to proliferate therein, and/or can proliferate on the surface of the collagen gel. For example, when a combination of the collagen sponge and the collagen gel is used as a scaffold substrate, the cell can be present between the pores of the porous structure of the collagen sponge together with the collagen gel, and can three-dimensionally proliferate in, and/or on the surface of, the collagen sponge. Although the incubator for the three-dimensional culture is not particularly limited, for example, an incubator subjected to cell adhesion treatment (e.g., a culture flask, a petri dish, or a multi-well plate) may be used. Specifically, for example, a cell culture plate (VTC-P96, VIOLAMO) may be used.

### (Scaffold Substrate)

The scaffold substrate to be used in the three-dimensional culture of the present invention is not particularly limited as long as the substrate is a material that provides the effect of the present invention and may be used in the application of cartilage repair. However, a collagen substrate containing collagen as a main raw material is preferred. Although the form of the collagen substrate is not particularly limited, for example, a collagen sponge, a collagen gel, a collagen sheet, or a combination thereof is available. Of those, the collagen sponge and/or the collagen gel is preferred, and a combination of the collagen sponge and the collagen gel is more preferred.

### (Collagen Sponge)

The collagen sponge is a porous construct having a plurality of pore structures. The collagen sponge to be used in the present invention is preferably produced by directly subjecting a collagen solution in a state in which no collagen fiber is formed to freeze drying treatment. Accordingly, collagen molecules are not aligned in one direction, but are randomly aligned, and hence the sponge can obtain sufficient strength and sufficient handleability in the solution or in a living body.

The collagen sponge to be used in the present invention may be a collagen sponge that have been subjected to insolubilization treatment with a chemical cross-linking agent. The insolubilization treatment increases the physical strength of the collagen sponge, and extends a remaining period in a tissue into which the collagen sponge has been transplanted. The insolubilization treatment cross-links collagen molecules in a random alignment, and increases the mechanical strength of the collagen sponge in all directions.

The insolubilization treatment with a chemical cross-linking agent is performed by bringing the whole of a freeze-dried product of a collagen solution (hereinafter sometimes referred to as “dried collagen product”) into contact with the chemical cross-linking agent without deforming the dried collagen product. Examples of the chemical cross-linking agent include a water-soluble chemical cross-linking agent and a vaporable chemical cross-linking agent. The insolubilization treatment with a water-soluble chemical cross-linking agent may be performed by immersing the dried collagen product in the water-soluble cross-linking agent. The insolubilization treatment with a vaporable chemical cross-linking agent may be performed by placing the dried collagen product and the chemical cross-linking agent (e.g., a formalin solution) in a sealed container. The chemical cross-linking agent is preferably the water-soluble chemical cross-linking agent.

The collagen sponge to be used in the present invention preferably has a uniform pore structure. The mean pore diameter of the collagen sponge falls within the range of 1 μm or more and less than 300 μm, preferably the range of 5 μm or more and 200 μm or less. In addition, the pore diameter standard deviation of the collagen sponge is preferably 20 μm or less, more preferably 15 μm or less, still more preferably 7 μm or less. In the collagen sponge of the present invention, a value obtained by dividing a value of the pore diameter standard deviation by the mean pore diameter (value of pore diameter standard deviation/mean pore diameter) is preferably 0.7 or less, more preferably 0.6 or less. The mean pore diameter and the standard deviation may be calculated by randomly selecting a plurality of (e.g., 100) pores from the surface of the collagen sponge, measuring the long diameter of each of the pores, and defining the long diameter as the diameter of the pore. The collagen sponge to be used in the present invention can obtain high tensile strength in any direction because the mean diameter of its pores is preferably smaller than that of a conventional collagen sponge, and hence the sponge has a dense pore structure and is uniform. When the mean diameter is 1 μm or less, a property required in cartilage treatment is not obtained because no cells can infiltrate into the pores.

The collagen sponge to be used in the present invention may be a freeze-dried product of an acidic collagen solution. The acidic collagen solution is an acidic solution in which collagen is dissolved in a solvent. A preferred pH is a pH of 1 or more and 4.5 or less, more preferably a pH of 2 or more and 4 or less, still more preferably a pH of 2.5 or more and 3.5 or less. The acidic collagen solution may contain an additive. The additive is suitably, for example, an additive that does not promote fibrillogenesis of the collagen. The acidic collagen solution is uniform throughout the entirety of the solution, has a uniform dispersed state of collagen molecules, and does not have collagen fibrils formed therein. The freeze-dried product of the acidic collagen solution is a product obtained by freeze-drying the acidic collagen solution, has a uniform pore structure, has more uniform compressive strength and tensile strength, and has properties of being strong against tension from any direction and being strong against compression from any direction. The freeze-dried product does not have collagen fibrils formed therein, and has collagen molecules aligned randomly instead of being aligned in one direction.

One aspect of the collagen sponge to be used in the present invention is a collagen sponge obtained as follows: a collagen solution subjected to stirring and deaeration treatment is freeze-dried to provide a porous construct having a pore structure; and the construct is subjected to insolubilization treatment with a chemical cross-linking agent. Such collagen sponge may be produced by a production method described in WO 2018/123814 A1, and may be, for example, an atelocollagen sponge (MIGHTY, KKN-CSM-50) manufactured by Koken Co., Ltd.

As the collagen to be used as a material for the collagen substrate to be used in the present invention, there may be used: insoluble collagen collected from a living body tissue, such as tendon collagen derived from the Achilles tendon or collagen derived from the skin; or soluble collagen or solubilized collagen, such as enzyme-solubilized collagen (atelocollagen), alkali-solubilized collagen, acid-soluble collagen, or salt-soluble collagen, and in particular, the atelocollagen is preferred. The species of an animal from which the collagen is derived is not particularly limited, and any collagen having such a denaturation temperature that the collagen does not denature by heat during culture may be used without problems. Specifically, there may be used collagen derived from a mammal, such as cattle or a pig, collagen derived from a bird such as chicken, or collagen derived from fish, such as tuna or tilapia. Recombinant collagen may also be used. In the collagen to be used as a material for the collagen sponge of the present invention, a side chain of a constituent amino acid of the collagen may be subjected to chemical modification. A specific example thereof is collagen subjected to acylation, such as acetylation, succinylation, or phthalation, alkylation, such as methylation or ethylation, or esterification.

The composition for cartilage repair of the present invention including the above-mentioned collagen sponge may be used in cartilage treatment, and is suitable for being implanted as a reinforcement or substitute for a cartilage tissue into a living body. In the culture, the cells may be cultured while a load similar to that to be applied to a cartilage tissue of a living body is applied. The collagen sponge has tensile strength and compressive strength (stress) equivalent to those of a cartilage tissue into which the collagen sponge is to be implanted, and has no unevenness in structure and strength, and hence can reduce a mechanical load to be applied to a tissue around the tissue into which the collagen sponge is implanted, and can also maintain a pore structure for allowing cells to infiltrate thereinto.

A method of treating a joint including using the composition for cartilage repair of the present invention including the above-mentioned collagen sponge is not particularly limited, but an example thereof is described below. First, small holes (e.g., at two or three sites) are made in a skin surface surrounding a joint in need of treatment, and a trocar is inserted into the joint. Insides of the joint and the trocar are filled with physiological saline, and an endoscope is passed through the inside of the trocar to gain direct access to a surgical site. A reinforcement or substitute including the composition for cartilage repair of the present invention is threaded with a suture thread and soaked in physiological saline. Under the state in which the substitute is immersed in physiological saline, the substitute is passed through the inside of the trocar to be transported into the joint by pulling the suture thread of the substitute. The substitute is implanted into the damaged site of the cartilage tissue of the joint, and is sewn with the suture thread. After that, the trocar and the endoscope are removed from the joint, and the holes in the skin surface are closed.

The composition for cartilage repair of the present invention including the above-mentioned collagen sponge has high biocompatibility, and hardly causes an antigen-antibody reaction when transplanted. In addition, the composition is decomposed by phagocytes or the like to disappear in a certain period of time. When substituted in a defective portion of cartilage, in particular, a meniscus defective portion, the substitute allows surrounding cells to infiltrate thereinto, is decomposed over time, and allows the reconstruction of the tissue along with the decomposition. The cells that have infiltrated into the collagen sponge secrete an ex vivo matrix to regenerate the tissue. Even after the decomposition of the collagen sponge, the cells and the secreted matrix remain, resulting in a state in which the tissue has been regenerated.

The collagen sponge to be used in the present invention is particularly suited for use as a reinforcement for a meniscus. The meniscus is a tissue sandwiched between the articular cartilages of a femur and a tibia, is subjected to strong forces from above and below, and changes its shape in response to the forces. When merely press-fitted to a defective portion of the meniscus, a substitute formed of a collagen sponge falls off, and hence suture is required. The collagen sponge to be used in the present invention can maintain its tensile strength even when exposed to a body fluid or water, and has such physical strength that, even when the defective portion is sutured, the collagen sponge is not broken from a portion threaded with a suture thread to fall off, and can endure the suture of the tissue. That is, the collagen sponge can be used as a substitute in treatment involving reconstructing the meniscus (suture or substitution). The collagen sponge is also excellent in compressive strength, and hence the pore structure therein does not collapse and allows surrounding cells to infiltrate thereinto, and besides, the collagen sponge does not apply a load (physical stimulus) to a surrounding tissue when transplanted into the meniscus.

The composition for cartilage repair of the present invention including the above-mentioned collagen sponge is particularly preferably used as a substitute for substitution for the total removal, total absence, subtotal removal, or partial absence of the inner edge of the meniscus. The substitute for the inner edge needs to have a maximum size of a length of 30 mm. The collagen sponge to be used in the present invention can provide a substitute that, even when having a size of a length of 30 mm, has high biocompatibility, has a uniform pore structure and uniform compressive strength, and has tensile strength allowing suture. The collagen sponge to be used in the present invention may be used for surgery under arthroscopic view. The collagen sponge to be used in the present invention has strength capable of enduring suture even when impregnated with water in blood, a body fluid, physiological saline, or the like. In the surgery under arthroscopic view, the collagen sponge to be used in the present invention can be passed through the inside of a trocar by threading the collagen sponge infiltrated with physiological saline with a suture thread and pulling the suture thread, and can be sutured to a tissue in the state of being immersed in physiological saline.

The composition for cartilage repair of the present invention including the above-mentioned collagen sponge is particularly suited for the treatment of the meniscus among cartilage tissues. Accordingly, it is important that the shape of the collagen sponge have a thickness (height) of 1 mm or more and 10 mm or less, preferably 3 mm or more and 5 mm or less. It is preferred that the shape of the collagen sponge have a length of 1 mm or more and 50 mm or less, preferably 5 mm or more and 30 mm or less, and a width of 1 mm or more and 50 mm or less, preferably 5 mm or more and 30 mm or less. In addition, the collagen sponge to be used in the present invention can be cut and processed so as to have a shape matching the size of a defect of each tissue, and hence does not entail an operation such as joining of a plurality of substitutes together. When the collagen sponge is used for substitution for the total removal, total absence, subtotal removal, or partial absence of the meniscus, the collagen sponge preferably has a disc shape or a crescent shape. When the collagen sponge is used for regeneration treatment of the meniscus, it is conceived that the collagen sponge allows the meniscus to be repaired to its original size, and hence the risk of developing gonarthrosis can be reduced.

### (Production Method for Composition for Cartilage Repair)

The composition for cartilage repair of the present invention may be produced by a production method including a step of three-dimensionally culturing an umbilical cord tissue-derived cell. The culture period of the three-dimensional culture is not particularly limited as long as the umbilical cord tissue-derived cell can maintain its undifferentiation property during the period. However, for example, the period may be from a time point immediately after the start of the culture to 8 weeks thereafter, from 1 day to 8 weeks thereafter, from a time point immediately after the start of the culture to 6 weeks thereafter, from 1 day to 6 weeks thereafter, from a time point immediately after the start of the culture to 5 weeks thereafter, or from 1 day to 5 weeks thereafter. The period is preferably from a time point immediately after the start of the culture to 28 days thereafter, preferably from 1 day to 28 days thereafter, preferably from 2 days to 28 days thereafter, preferably from 3 days to 28 days thereafter, preferably from 4 days to 28 days thereafter, preferably from 5 days to 28 days thereafter, preferably from 6 days to 28 days thereafter, preferably from 7 days to 28 days thereafter, preferably from 14 days to 28 days thereafter. The period is more preferably from a time point immediately after the start of the culture to 14 days thereafter, more preferably from 1 day to 14 days thereafter, more preferably from 2 days to 14 days thereafter, more preferably from 3 days to 14 days thereafter, more preferably from 4 days to 14 days thereafter, more preferably from 1 day to 7 days thereafter, more preferably from 2 days to 7 days thereafter, more preferably from 3 days to 7 days thereafter, more preferably from 4 days to 7 days thereafter, more preferably from a time point immediately after the start of the culture to 4 days thereafter, more preferably from 1 day to 4 days thereafter, more preferably from 2 days to 4 days thereafter, more preferably from 3 days to 4 days thereafter, more preferably 4 days thereafter. In one preferred aspect of the present invention, the step of three-dimensionally culturing the umbilical cord tissue-derived cell includes a step of culturing the umbilical cord tissue-derived cell in the presence of a scaffold substrate.

(Step of culturing Umbilical Cord Tissue-derived Cell in Presence of Scaffold Substrate)

In one aspect of the present invention, the step of culturing the umbilical cord tissue-derived cell in the presence of the scaffold substrate is as described below. The umbilical cord tissue-derived cells are suspended in a proliferation medium such as DMEM containing 10% FBS and penicillin-streptomycin to prepare a cell suspension. The umbilical cord tissue-derived cells in the cell suspension are inoculated into the scaffold substrate. Although a cell density is not particularly limited as long as the cells can proliferate, the inoculation may be performed so that the cell density may be, for example, from 1.0×10² cells to 1.0×10⁵ cells, preferably from 5.0×10² cells to 2.0×10⁴ cells, more preferably from 1.0×10³ cells to 1.5×10⁴ cells, particularly preferably from 1.5×10³ cells to 8.5×10³ cells per 1 mm³ of the scaffold substrate. The inoculated cells are cultured under static conditions at 37° C.±1° C. in 5% CO₂ for 1 day or more. The medium is replaced with a new one once per from 1 day to 5 days, preferably once per from 2 days to 3 days. The cell suspension may be mixed with a collagen gel. When the collagen gel is mixed, the collagen gel is mixed so as to have a final concentration of from 0.001% to 50%, preferably from 0.01% to 10%, more preferably from 0.05% to 5%, particularly preferably from 0.1% to 1%, most preferably 0.5%.

The production method for a composition for cartilage repair of the present invention may further include a step of subculturing the umbilical cord tissue-derived cell, preferably a step of subculturing the umbilical cord tissue-derived cell up to from a second generation to a fifth generation before the step of three-dimensionally culturing the umbilical cord tissue-derived cell. A medium that may be used in the maintenance and culture of the umbilical cord tissue-derived cell is not particularly limited because the umbilical cord tissue-derived cell only needs to be capable of being cultured therein. For example, however, the following medium may be used as a main medium: the medium contains Dulbecco Modified Eagle's Medium as a raw material, preferably as a main component, and a serum such as fetal bovine serum and antibiotics, such as penicillin and streptomycin, are appropriately incorporated thereinto. For example, a medium component described in Sci Rep. 2021 Jan. 19; 11 (1): 1757. may be used as a medium component.

The split ratio of the umbilical cord tissue-derived cells may be set to from 1:2 to 1:5. The medium may be replaced with a new one once per from 1 day to 7 days, preferably once per from 2 days to 3 days.

The production method for a composition for cartilage repair of the present invention may include a step of producing and storing a master cell before the step of three-dimensionally culturing the umbilical cord tissue-derived cell in the presence of the scaffold substrate, and/or before or after the step of subculturing the umbilical cord tissue-derived cell.

### (Function and Property of Umbilical Cord Tissue-Derived Cell for Forming Three-Dimensionally Cultured Product)

The umbilical cord tissue-derived cell for forming the three-dimensionally cultured product in the composition for cartilage repair of the present invention has at least one of the following features.
(1) The cell proliferation potency of the cell in its three-dimensional culture is higher than that of a cell for forming a cultured product obtained by the three-dimensional culture of a synovial tissue-derived cell and/or a meniscus-derived cell. A case in which the proliferation potency of an undifferentiated cell having an ability to differentiate into a chondrocyte is high may be advantageous to cartilage repair and particularly advantageous to the repair of a meniscus requiring high strength because the repair of the chondrocyte is promoted.
(2) The cell is low in expression levels of one or a plurality of kinds of matrix protease genes selected from the group consisting of: MMP1; MMP2; MMP3; MMP9; MMP13; and MT1-MMP as compared to the cell for forming the cultured product obtained by the three-dimensional culture of the synovial tissue-derived cell and/or the meniscus-derived cell. A matrix protease is known as an extracellular matrix-degrading enzyme, and a case in which its expression level is low may be advantageous to cartilage repair and particularly advantageous to the repair of a meniscus requiring high strength because the degradation of a cartilage tissue is suppressed. An amino acid sequence encoded by the MMP1 gene is disclosed in, for example, NCBI Reference Sequence: NP_002412.1, and a mRNA sequence encoded by the gene is disclosed in, for example, NCBI Reference Sequence: NM_002421.4. An amino acid sequence encoded by the MMP2 gene is disclosed in, for example, NCBI Reference Sequence: NP_004521.1, and a mRNA sequence encoded by the gene is disclosed in, for example, NCBI Reference Sequence: NM_004530.6. An amino acid sequence encoded by the MMP3 gene is disclosed in, for example, NCBI Reference Sequence: NP_002413.1, and a mRNA sequence encoded by the gene is disclosed in, for example, NCBI Reference Sequence: NM_002422.5. An amino acid sequence encoded by the MMP9 gene is disclosed in, for example, NCBI Reference Sequence: NP_004985.2, and a mRNA sequence encoded by the gene is disclosed in, for example, NCBI Reference Sequence: NM_004994.3. An amino acid sequence encoded by the MMP13 gene is disclosed in, for example, NCBI Reference Sequence: NP_002418.1, and a mRNA sequence encoded by the gene is disclosed in, for example, NCBI Reference Sequence: NM_002427.4. An amino acid sequence encoded by the MTI-MMP gene is disclosed in, for example, NCBI Reference Sequence: NP_004986.1, and a mRNA sequence encoded by the gene is disclosed in, for example, NCBI Reference Sequence: NM_004995.4.
(3) The cell is high in expression levels of one or two kinds of integrin a subunit genes ITGA1 and ITGA2 as compared to the cell for forming the cultured product obtained by the three-dimensional culture of the synovial tissue-derived cell and/or the meniscus-derived cell. An integrin a subunit is known to form an integrin, which is involved in cell adhesion and signal transduction through a cell surface, together with an integrin β1 subunit. Of the integrin a subunits, the ITGA1 and the ITGA2 are each known to be involved in a collagen bond, and a case in which their expression levels are high may be advantageous to cartilage repair and particularly advantageous to the repair of a meniscus requiring high strength because a bond between a cell and an extracellular matrix is reinforced. An amino acid sequence encoded by the ITGA1 gene is disclosed in, for example, NCBI Reference Sequence: NP_852478.1, and a mRNA sequence encoded by the gene is disclosed in, for example, NCBI Reference Sequence: NM_181501.2. An amino acid sequence encoded by the ITGA2 gene is disclosed in, for example, NCBI Reference Sequence: NP_002194.2, and a mRNA sequence encoded by the gene is disclosed in, for example, NCBI Reference Sequence: NM_002203.4.
(4) The cell is low in expression levels of one or two kinds of integrin a subunit genes ITGA10 and ITGA11 as compared to the cell for forming the cultured product obtained by the three-dimensional culture of the synovial tissue-derived cell and/or the meniscus-derived cell. An amino acid sequence encoded by the ITGA10 gene is disclosed in, for example, NCBI Reference Sequence: NP_003628.2, and a mRNA sequence encoded by the gene is disclosed in, for example, NCBI Reference Sequence: NM_003637.5. An amino acid sequence encoded by the ITGA11 gene is disclosed in, for example, NCBI Reference Sequence: NP_001004439.1, and a mRNA sequence encoded by the gene is disclosed in, for example, NCBI Reference Sequence: NM 001004439.2.
(5) The cell is high in expression levels of one or two kinds of Oct4 and Nanog genes as compared to an umbilical cord tissue-derived cell cultured in the absence of the scaffold substrate. The Oct4 and the Nanog are each known as a gene involved in the promotion of self-replicability and the maintenance of an undifferentiation property, and a case in which their expression levels are high may be advantageous to cartilage repair. An amino acid sequence encoded by the Oct4 gene is disclosed in, for example, NCBI Reference Sequence: NP_002692.2, and a mRNA sequence encoded by the gene is disclosed in, for example, NCBI Reference Sequence: NM 002701.6. An amino acid sequence encoded by the Nanog gene is disclosed in, for example, NCBI Reference Sequence: NP_079141.2, and a mRNA sequence encoded by the gene is disclosed in, for example, NCBI Reference Sequence: NM_024865.4.

The amino acid sequences and the mRNA sequences set forth in the NCBI Reference Sequence numbers listed above are examples, and the present invention is not limited thereto.

### (Utilization and Application of Composition for Cartilage Repair)

The composition for cartilage repair of the present invention is utilized in the repair of a bone tissue by autotransplantation, isotransplantation, allotransplantation, or xenotransplantation. An application method is, for example, parenteral administration or local administration. More preferably, implantation, loading, or injection into, or application to, a damaged or defected cartilage tissue may be adopted. The composition for cartilage repair of the present invention may be widely utilized in the application of the repair of a cartilage tissue, and may be utilized in, for example, a cartilage-reconstructing substrate or a cell preparation for treating cartilage damage. The composition may be utilized in combination with known cartilage treatment, such as a cartilage graft, an autologous cultured cartilage, an artificial cartilage, an artificial joint, or an artificial meniscus. Herein, the cartilage damage is not particularly limited, and includes, for example, meniscus damage, traumatic cartilage deficiency, osteochondritis dissecans, arthrosis deformans, rheumatoid arthritis, an anterior cruciate ligament injury, patellar dislocation, and avascular necrosis of a medial femoral condyle. The composition for cartilage repair of the present invention may be not only applied to hitherto performed cartilage treatment, but also used for cartilage treatment to be developed in the future, in particular, meniscus regeneration treatment or intervertebral disc regeneration treatment.

Although a cartilage to which the composition for cartilage repair of the present invention is to be applied is not particularly limited, examples thereof may include a meniscus, an articular cartilage, an intervertebral disc, a costal cartilage, a tracheal cartilage, a bronchial cartilage, a nasal cartilage, a thyroid cartilage, a pubic symphysis, an articular disc, an epiphyseal plate, an auricular cartilage, a meatal cartilage, an epiglottic cartilage, a laryngeal cartilage, and an articular lip. When the scaffold substrate of the three-dimensionally cultured product in the composition for cartilage repair of the present invention includes a collagen sponge, the cartilage to which the composition for cartilage repair of the present invention is to be applied is preferably a meniscus or an intervertebral disc, particularly preferably a meniscus.

A subject to which the composition for cartilage repair of the present invention is to be administered is typically a mammalian animal. Although the mammalian animal is not particularly limited, examples thereof may include: experimental animals including rodents, such as a mouse, a rat, a hamster, and a guinea pig, and a rabbit; domestic animals, such as a pig, cattle, a goat, a horse, a sheep, and a mink; pets, such as a dog and a cat; and primates, such as a human, a monkey, a crab-eating macaque, a rhesus macaque, a marmoset, an orangutan, and a chimpanzee. Of those, a human is more preferred.

The composition for cartilage repair of the present invention includes a three-dimensionally cultured product formed of an effective dose of the umbilical cord tissue-derived cells. The term “effective dose” as used herein means a dose effective in preventing and/or treating cartilage damage. Such effective dose is appropriately regulated in accordance with the severity of the cartilage damage, and the medical factors of a patient and the like. The composition for cartilage repair of the present invention may include the umbilical cord tissue-derived cells in an amount of, for example, from 1,000 cells/mm³ to 100,000 cells/mm³, from 1,000 cells/mm³ to 10,000 cells/mm³, or from 50,000 cells/mm³ to 100,000 cells/mm³. The composition includes the cells in an amount of preferably from 5,000 cells/mm³ to 70,000 cells/mm³, more preferably from 10,000 cells/mm³ to 50,000 cells/mm³.

### (Other Component)

The composition for cartilage repair of the present invention may include any other component as required in addition to the three-dimensionally cultured product formed of the umbilical cord tissue-derived cell. For example, the composition may include a pharmaceutically acceptable carrier or an excipient. Specific examples thereof include a basic medium used for the production of the composition for cartilage repair, physiological saline, and a buffer solution. Further, dextrose, hyaluronic acid, polyethylene glycol, polyvinyl alcohol, or carboxymethylcellulose, or a combination thereof may be incorporated into the composition for cartilage repair of the present invention.

### EXAMPLES

The present invention is specifically described below by way of Examples and Comparative Examples described for deepening the understanding of the present invention. Needless to say, however, these examples do not limit the scope of the present invention.

### (Example 1) Isolation and Passage of Human Umbilical Cord Tissue-derived Mesenchymal Stem Cell

In this Example, the isolation, maintenance, and passage of a human umbilical cord tissue-derived mesenchymal stem cell to be used in subsequent Examples are described.

Human umbilical cord tissue-derived mesenchymal stem cells were isolated by using umbilical cord tissues derived from patients (six persons) for whom an approval by an ethics committee in Osaka University had been obtained and from whom consent had been obtained.

### 1. Isolation of Human Umbilical Cord Tissue-derived Mesenchymal Stem Cells

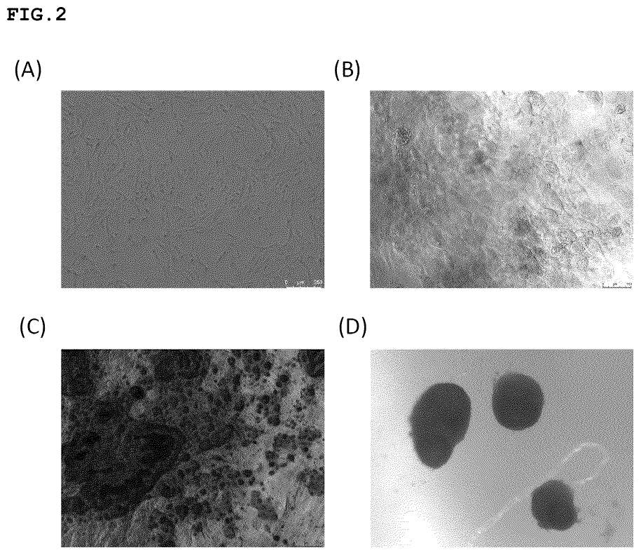

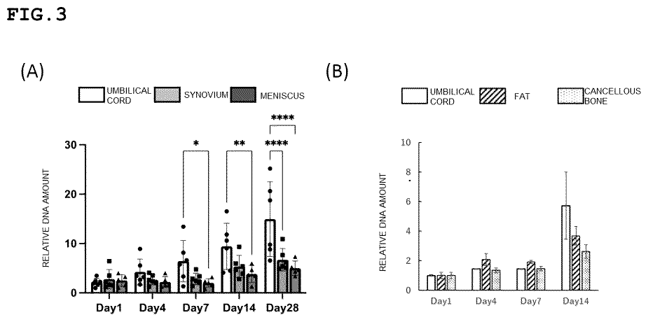

The human umbilical cord tissue-derived mesenchymal stem cells to be used in this Example were isolated by using the following two approaches: (1) a cell dispersion method including using an enzyme (protease); and (2) an explant method including culturing a physically cut tissue to provide a cell. The cells isolated by the cell dispersion method were used in a test whose results were shown in FIG. 2B, and the cells isolated by the explant method were used in tests whose results were shown in FIG. 2A and FIG. 3. The cell dispersion method is in conformity with a previous report (Kanamoto T et al., Scientific Reports 2021). Collagenase (Animal Origin Free)-B LS004145 (Funakoshi Company, Ltd./Worthington Biochemical Corporation) or STEMxyme 2 STZ2 (Funakoshi Company, Ltd./Worthington Biochemical Corporation) was used as the protease.

### (1) Cell Isolation by Cell Dispersion Method

The umbilical cord tissues that had been aseptically collected were washed with phosphate buffered saline (PBS), and their arteriovenous tissues were removed under direct vision or under a stereomicroscope. The residues were finely minced, and were then treated in Dulbecco Modified Eagle's Medium (DMEM) having added thereto 1% penicillin/streptomycin (Thermo Fisher Scientific, Inc.) containing the 0.1% protease at 37° C. for from 3 hours to 6 hours. The treated products were filtered with a 100-micrometer filter, and were then centrifuged (at from 1,000 rpm to 3,000 rpm) to provide the cells.

### (2) Cell Isolation by Explant Method

The umbilical cord tissues that had been aseptically collected were washed with PBS, and their arteriovenous tissues were removed under direct vision or under a stereomicroscope. The residues were finely minced, and were then cultured in DMEM containing 10% FBS and 1% penicillin/streptomycin through use of a TCPS dish as a culture container. Thus, adherent cells that had migrated to the outside of the tissues were obtained.

### 2. Maintenance and Passage of Human Umbilical Cord Tissue-derived Mesenchymal Stem Cells

The isolated cells were suspended in DMEM containing 10% fetal bovine serum (FBS) (Nichirei Biosciences Inc.) and 1% penicillin/streptomycin in a cell culture dish (VTC-D150/100, VIOLAMO). The cells were subjected to static culture through use of a CO₂ incubator (MCO-230/170AICUVH, PHC Corporation) under the conditions of 37° C. and 5% CO₂. The cells in from a second passage to a fifth passage were used in three-dimensional culture to be described later (Example 2). The medium of the human umbilical cord tissue-derived mesenchymal stem cells was replaced with a new one once per from 2 days to 3 days. Cells for passage were inoculated at passage intervals of from 3 days to 7 days and at a split ratio between 1:2 and 1:5. The cells (P2 to P5) after 2 to 5 passages were used in the three-dimensional culture to be described later (Example 2).

### 3. Identification of Mesenchymal Stem Cells (MSCs)

The human umbilical cord tissue-derived mesenchymal stem cells to be used in this Example were identified as mesenchymal stem cells by (1) the expression profile of a cell surface marker and (2) a differentiation ability evaluation.

### (1) Expression Profile of Cell Surface Marker

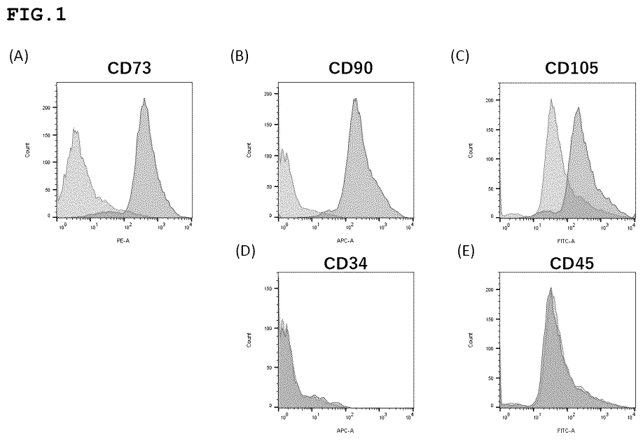

The human umbilical cord tissue-derived cells (P3) in the third passage during their plate culture were stained with a PE-labeled anti-CD73 antibody, an APC-labeled anti-CD90 antibody, a FITC-labeled anti-CD105 antibody, an APC-labeled anti-CD34 antibody, a FITC-labeled anti-CD45 antibody, a PE-labeled anti-IgG antibody, an APC-labeled anti-IgG antibody, and a FITC-labeled anti-IgG antibody (the antibodies were each manufactured by BioLegend, Inc.). The stained cells were subjected to measurement with Canto™ II (manufactured by Becton, Dickinson and Company) by a flowcytometry method. The relative expression of a MSC marker (CD73, CD90, CD105, CD34, or CD45) was evaluated by displaying its histogram while superimposing the histogram of an isotype control (anti-IgG antibody) thereon. The results are shown in FIG. 1. As shown in FIG. 1A to FIG. 1E, the ratios of CD73 (+), CD90 (+), and CD105 (+) cells were high, and substantially no CD34 (+) and CD45 (+) cells were present. Accordingly, it was recognized from the expression profiles of the surface cell markers that the human umbilical cord tissue-derived cells of this Example were positive for the CD73, the CD90, and the CD105, and were negative for the CD34 and the CD45 (CD73+/CD90+/CD105+/CD34−/CD45−), and hence each had a feature of a MSC.

### (2) Differentiation Ability Evaluation

The human umbilical cord tissue-derived cells in the third passage of the plate culture were evaluated for their abilities to differentiate into an adipocyte, an osteoblast, and a chondrocyte with a PromoCell (trademark) differentiation kit in accordance with the protocol of the kit. With regard to the evaluation for an ability to differentiate into an adipocyte, the cells were cultured in an adipocyte differentiation medium for 13 days, and were then stained with Oil Red and observed under an optical microscope. With regard to the evaluation for an ability to differentiate into an osteoblast, the cells were cultured in an osteoblast differentiation medium for 13 days, and were then stained with Alizarin Red and observed under an optical microscope. With regard to the evaluation for an ability to differentiate into a chondrocyte, the cells were cultured in a chondrocyte differentiation medium for 13 days, and were then stained with Alcian Blue and observed under an optical microscope. The results are shown in FIG. 2. As shown in FIG. 2B to FIG. 2D, it was recognized that each of the human umbilical cord tissue-derived cells of this Example had abilities to differentiate into an adipocyte, an osteoblast, and a chondrocyte, and hence had multipotency serving as a feature of a MSC.

### (Comparative Example 1) Isolation and Passage of Human Synovial Tissue-derived Mesenchymal Stem Cell

Human synovial tissue-derived mesenchymal stem cells were isolated by using synovial tissues derived from patients (six persons) for whom an approval by an ethics committee in Osaka University had been obtained and from whom consent had been obtained.

The human synovial tissue-derived mesenchymal stem cells to be used in this Comparative Example were isolated from the synovial tissues, which had been aseptically collected, by using a cell dispersion method in the same manner as in Example 1 described above, and the cells after 2 to 5 passages were used in three-dimensional culture.

### (Comparative Example 2) Isolation and Passage of Human Meniscus Tissue-derived Mesenchymal Stem Cell

Human meniscus tissue-derived mesenchymal stem cells were isolated by using meniscus tissues derived from patients (five persons) for whom an approval by an ethics committee in Osaka University had been obtained and from whom consent had been obtained.

The human meniscus tissue-derived mesenchymal stem cells to be used in this Comparative Example were isolated from the meniscus tissues, which had been aseptically collected, by using a cell dispersion method in the same manner as in Example 1 described above, and the cells after 2 to 5 passages were used in three-dimensional culture.

### (Comparative Example 3) Isolation and Passage of Human Fat Tissue-derived Mesenchymal Stem Cell

Human fat tissue-derived mesenchymal stem cells were isolated by using a fat tissue derived from a patient (one person) for whom an approval by an ethics committee in Osaka University had been obtained and from whom consent had been obtained.

The human fat tissue-derived mesenchymal stem cells to be used in this Comparative Example were isolated from the fat tissue, which had been aseptically collected, by using an explant method in the same manner as in Example 1 described above, and the cells after 2 to 5 passages were used in the following Examples.

### (Comparative Example 4) Isolation and Passage of Human Cancellous Bone Tissue-derived Mesenchymal Stem Cell

Human cancellous bone tissue-derived mesenchymal stem cells were isolated by using a cancellous bone tissue derived from a patient (one person) for whom an approval by an ethics committee in Osaka University had been obtained and from whom consent had been obtained.

The human cancellous bone tissue-derived mesenchymal stem cells to be used in this Comparative Example were isolated from the cancellous bone tissue, which had been aseptically collected, by using an explant method in the same manner as in Example 1 described above, and the cells after 2 to 5 passages were used in three-dimensional culture.

### (Example 2) Three-dimensional Culture of Mesenchymal Stem Cells derived from Respective Tissues such as Umbilical Cord Tissue-derived Mesenchymal Stem Cells

In this Example, the three-dimensional culture of the mesenchymal stem cells derived from the various tissues, which had been isolated, maintained, and passaged in Example 1 and Comparative Examples 1 to 4, was performed.

1. Inoculation of Cells into Atelocollagen Sponge

An atelocollagen sponge having a diameter of 5 mm and a thickness of 3 mm (MIGHTY, KKN-CSM-50, Koken Co., Ltd.) was used as a scaffold substrate for the three-dimensional culture. Atelocollagen mainly includes bovine skin-derived type I collagen, and the sponge has continuous holes each having a diameter of from 30 μm to 200 μm, and can withstand a compressive load of up to 40 kPa. The inoculation of the cells into the atelocollagen sponge was performed by the following two methods: a case in which an atelocollagen gel was used; and a case in which the gel was not used. The mesenchymal stem cells derived from the various tissues, which had been isolated, maintained, and passaged in Example 1 and Comparative Examples 1 to 4, were suspended in a proliferation medium (DMEM containing 10% FBS and penicillin-streptomycin), and the suspension was mixed with a 1% atelocollagen gel (Koken Co., Ltd.) whose amount was equal to that of the suspension. Thus, a 0.5% collagen gel-containing cell suspension was produced. The atelocollagen sponges were loaded one by one into each well of a 96-well cell culture plate (VTC-P96, VIOLAMO), and the produced cell suspension was carefully dropped at from 50 μL/well to 70 μL/well (from 1×10⁵ cells/well to 5×10⁵ cells/well) so that the sponges were immersed in the suspension.

### 2. Three-Dimensional Culture

The 96-well plate into which the various cells had been inoculated was left to stand still under the conditions of 37° C. and 5% CO₂, and the culture of the cells with a CO₂ incubator (MCO-230/170AICUVH, PHC Corporation) was continued for 28 days. The medium was replaced with a new one once per from 2 days to 3 days.

### (Example 3) Measurement of Relative DNA Amounts of Three-dimensionally Cultured Mesenchymal Stem Cells derived from Various Tissues

In this Example, the relative DNA amounts of the respective three-dimensionally cultured cells were measured. In the three-dimensional culture of Example 2, a three-dimensionally cultured product containing the mesenchymal stem cells derived from each of the various tissues and the atelocollagen sponge at each of the following time points after the inoculation of the cells into the atelocollagen sponge was collected: 1st, 4th, 7th, 14th, and 28th days. DNA was extracted with PureLink™ Genomic DNA Purification Kit (Thermo Fisher Scientific, Inc.) in accordance with the instruction of the manufacturer. A DNA concentration was determined with Qubit (trademark) 4.0 Fluorometer (Thermo Fisher Scientific, Inc.) by fluorometry. The results are presented as means and standard deviations (SDs). With regard to statistical processing, single comparison was performed by using Student's t-test, and multiple comparison was performed by using one-way analysis of variance (ANOVA) and post-hoc Tukey-Kramer's test. Excel Statistics (Social Survey Research Information Co., Ltd.) was used in statistical analysis.

The relative DNA amount of the umbilical cord tissue-derived mesenchymal stem cells at each of the following time points was equal to or more than that of the mesenchymal stem cells derived from any other tissue, and hence the umbilical cord tissue-derived mesenchymal stem cells showed cell proliferation potency equal to or higher than that of the mesenchymal stem cells derived from any other tissue (FIG. 3A and FIG. 3B): the 1st, 4th, 7th, 14th, and 28th days of the three-dimensional culture. As shown in FIG. 3A, as compared to the synovial tissue-derived mesenchymal stem cells that had heretofore been frequently used in the cell therapy of a joint tissue, the umbilical cord tissue-derived mesenchymal stem cells showed high cell proliferation potency on each of the 4th, 7th, 14th, and 28th days of the culture, and showed significantly high cell proliferation potency on the 28th day of the culture (****: p<0.001). In addition, as shown in FIG. 3A, also as compared to the meniscus tissue-derived mesenchymal stem cells, the umbilical cord tissue-derived mesenchymal stem cells showed high cell proliferation potency on each of the 4th, 7th, 14th, and 28th days of the culture, and showed significantly high cell proliferation potency on each of the 7th, 14th, and 28th days of the culture (*: p<0.05, **: p<0.01, ****: p<0.001). In addition, as shown in FIG. 3B, as compared to the fat tissue-derived mesenchymal stem cells and the cancellous bone tissue-derived mesenchymal stem cells, the umbilical cord tissue-derived mesenchymal stem cells showed comparable or high cell proliferation potency on each of the 1st, 4th, 7th, and 14th days of the culture. It was found from the foregoing that the cell proliferation potency of the umbilical cord tissue-derived mesenchymal stem cells in their three-dimensional culture was higher than that of the mesenchymal stem cells derived from any other tissue typified by the synovial tissue, and the high cell proliferation potency continued for at least 4 weeks from the initial stage of the culture.

### (Example 4) Gene Expression Analysis of Three-dimensionally Cultured Mesenchymal Stem Cells derived from Various Tissues

In this Example, the various gene expression levels of the respective three-dimensionally cultured cells were measured. The mesenchymal stem cells derived from the various tissues were three-dimensionally cultured in the same manner as in Example 2, and the three-dimensionally cultured product containing the mesenchymal stem cells derived from each of the various tissues and the atelocollagen sponge at the time point of the 4th day after the inoculation of the cells into the atelocollagen sponge was collected, followed by the measurement by a quantitative PCR. Total RNA was extracted with TRIzol (Invitrogen) and PureLink™ RNA Purification Kit (Thermo Fisher Scientific, Inc.), and reverse transcription into cDNA was performed with High-Capacity RNA-to-cDNA Kit (Thermo Fisher Scientific, Inc.). The quantitative PCR was performed with Power SYBR (trademark) Green Master Mix and QuantiStudio 7 Pro Real-Time PCR System (Thermo Fisher Scientific, Inc.). A sequence used in a primer is as shown in Table 1. The expression of a target gene was normalized with respect to the expression of glyceraldehyde-3-phosphate dehydrogenase (GAPDH) serving as a reference gene. The results are presented as means and standard deviations (SDs). With regard to statistical processing, single comparison was performed by using Student's t-test, and multiple comparison was performed by using one-way analysis of variance (ANOVA) and post-hoc Tukey-Kramer's test. Excel Statistics (Social Survey Research Information Co., Ltd.) was used in statistical analysis.

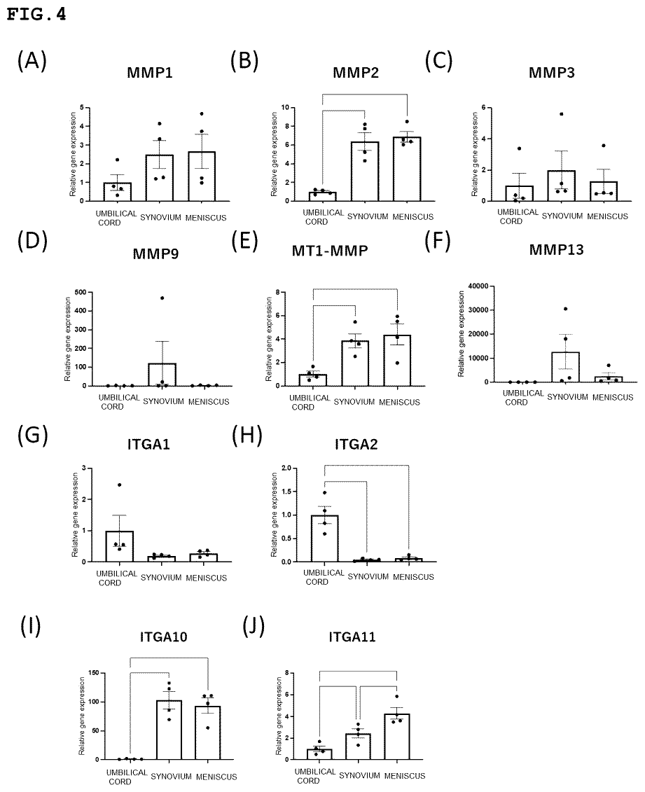

In the three-dimensional culture using the collagen sponge as a scaffold substrate, the umbilical cord tissue-derived MSCs showed gene expression different from that of the MSCs derived from any other tissue. The expression of each of integrin a subunits of types α1 and α2 tended to be high, and the expression of a matrix metalloprotease family gene tended to be low. More specifically, as compared to the synovial tissue-derived mesenchymal stem cells, the umbilical cord tissue-derived mesenchymal stem cells were low in expression of each of MMP1, MMP2, MMP3, MMP9, MT1-MMP, MMP13, ITGA10, and ITGA11 genes (FIG. 4A to FIG. 4F, FIG. 4I, and FIG. 4J), and were high in expression of each of ITGA1 and ITGA2 genes (FIG. 4G and FIG. 4H). In addition, also as compared to the meniscus tissue-derived mesenchymal stem cells, the umbilical cord tissue-derived mesenchymal stem cells were low in, or negative for, expression of each of the MMP1, MMP2, MMP3, MMP9, MT1-MMP, MMP13, ITGA10, and ITGA11 genes (FIG. 4A to FIG. 4F, FIG. 4I, and FIG. 4J), and were high in expression of each of the ITGA1 and ITGA2 genes (FIG. 4G and FIG. 4H).

TABLE 1

| Primer | Sequence | |
|---|---|---|
| MMP1 F | CCCAAAAGCGTGTGACAGTAAG | (SEQ ID NO: 1) |
| MMP1 R | CTTCCGGGTAGAAGGGATTTG | (SEQ ID NO: 2) |
| MMP2 F | GCACCCATTTACACCTACACCAA | (SEQ ID NO: 3) |
| MMP2 R | AGAGCTCCTGAATGCCCTTGA | (SEQ ID NO: 4) |
| MMP3 F | CGTGAGGAAAATCGATGCAG | (SEQ ID NO: 5) |
| MMP3 R | CTTCAGCTATTTGCTTGGGAAAG | (SEQ ID NO: 6) |
| MMP9 F | AGTCCACCCTTGTGCTCTTC | (SEQ ID NO: 7) |
| MMP9 R | TTTCGACTCTCCACGCATC | (SEQ ID NO: 8) |
| MT1-MMP1 F | TCAGGGCAGTGGATAGCGA | (SEQ ID NO: 9) |
| MT1-MMP1 R | GCCGGTTCTACCTTCAGCTTC | (SEQ ID NO: 10) |
| ITGA1 F | CAGCCCCACATTTCAAGTCGT | (SEQ ID NO: 11) |
| ITGA1 R | ACCTGTGTCTGTTTAGGACCA | (SEQ ID NO: 12) |
| ITGA2 F | GCAACTGGTTACTGGTTGGTT | (SEQ ID NO: 13) |
| ITGA2 R | GAGGCTCATGTTGGTTTTCATCT | (SEQ ID NO: 14) |
| ITGA10 F | CTTCAGTTCTGGGATATGTGCC | (SEQ ID NO: 15) |
| ITGA10 R | CCAGTCTTCGTAGGAAGGTCT | (SEQ ID NO: 16) |
| ITGA11 F | TCACGGACACCTTCAACATGG | (SEQ ID NO: 17) |
| ITGA11 R | CCAGCCACTTATTGCCACTGA | (SEQ ID NO: 18) |
| GAPDH F | TCTCTGCTCCTCCTGTTCGAC | (SEQ ID NO: 19) |
| GAPDH R | GTTGACTCCGACCTTCACCTTC | (SEQ ID NO: 20) |
| MMP13 F | CTTCCCAACCGTATTGATGC | (SEQ ID NO: 21) |
| MMP13 R | ACTTCTTTTGGAAGACCCAGTTC | (SEQ ID NO: 22) |

### (Example 5) Gene Expression Analysis of Plate-cultured Cells and Three-dimensionally Cultured Cells

In this Example, the expression levels of the undifferentiated marker genes of plate-cultured umbilical cord tissue-derived mesenchymal stem cells and three-dimensionally cultured umbilical cord tissue-derived mesenchymal stem cells were measured. The cells at the time point of the plate culture on a proliferative phase subcultured by the same method as that of Example 1 and the cells at the time point of the three-dimensional culture on the 4th day after the inoculation of the cells into the atelocollagen sponge (using the atelocollagen gel) by the same method as that of Example 2 were collected, and the measurement was performed by a quantitative PCR. Total RNA was extracted with TRIzol (Invitrogen) and PureLink™ RNA Purification Kit (Thermo Fisher Scientific, Inc.), and reverse transcription into cDNA was performed with High-Capacity RNA-to-cDNA kit (Thermo Fisher Scientific, Inc.). The quantitative PCR was performed with Power SYBR (trademark) Green Master Mix and QuantiStudio 7 Pro Real-Time PCR System (Thermo Fisher Scientific, Inc.). A sequence used in a primer is as shown in Table 2. The expression of a target gene was normalized with respect to the expression of glyceraldehyde-3-phosphate dehydrogenase (GAPDH) serving as a reference gene. The results are represented as means and standard deviations (SDs). With regard to statistical processing, single comparison was performed by using Student's t-test, and multiple comparison was performed by using one-way analysis of variance (ANOVA) and post-hoc Tukey-Kramer's test. Excel Statistics (Social Survey Research Information Co., Ltd.) was used in statistical analysis.

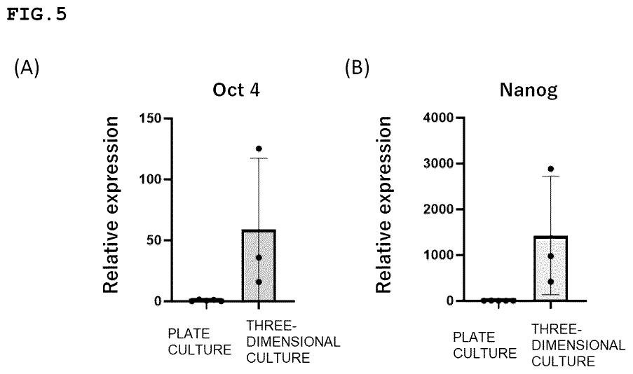

The umbilical cord tissue-derived MSCs at the time of the three-dimensional culture were high in expression levels of Oct4 and Nanog genes each serving as a gene involved in an undifferentiation property as compared to those at the time of the plate culture (FIG. 5A and FIG. 5B).

TABLE 2

| Primer | Sequence | |
|---|---|---|
| HS OCT4 F | CTCCTGGAGGGCCAGGAATC | (SEQ ID NO: 23) |
| HS OCT4 R | CCACATCGGCCTGTGTATAT | (SEQ ID NO: 24) |
| HS NANOG F | CAAAGGCAAACAACCCACTT | (SEQ ID NO: 25) |
| HS NANOG R | TCTGCTGGAGGCTGAGGTAT | (SEQ ID NO: 26) |

### INDUSTRIAL APPLICABILITY

The umbilical cord tissue-derived cell for forming the three-dimensionally cultured product in the composition for cartilage repair of the present invention has high cell proliferation potency in three-dimensional culture, has a feature suitable for cartilage repair, and may be utilized in autologous or allogeneic regenerative medicine. The composition for cartilage repair of the present invention may be widely utilized in the application of the repair of a cartilage tissue. For example, the composition may be utilized in an artificial cartilage, an artificial joint, an artificial meniscus, a graft, a cell preparation, an injection, or a cartilage substitute.

1. A composition for cartilage repair, comprising, as an active ingredient, a three-dimensionally cultured product formed of an umbilical cord tissue-derived cell.

2. The composition for cartilage repair according to claim 1, wherein an expression level of a matrix metalloprotease gene in the umbilical cord tissue-derived cell for forming the three-dimensionally cultured product is lower than an expression level of the gene in a synovial tissue-derived cell or a meniscus-derived cell.

3. The composition for cartilage repair according to claim 2, wherein the matrix metalloprotease gene is one or a plurality of kinds of genes selected from the group consisting of: MMP1; MMP2; MMP3; MMP9; MMP13; and MT1-MMP.

4. The composition for cartilage repair according to claim 1,
wherein expression levels of one or two kinds of ITGA1 and ITGA2 genes in the umbilical cord tissue-derived cell for forming the three-dimensionally cultured product are higher than an expression level of each of the genes in a synovial tissue-derived cell or a meniscus-derived cell, and/or
wherein expression levels of one or two kinds of ITGA10 and ITGA11 genes in the umbilical cord tissue-derived cell for forming the three-dimensionally cultured product are lower than an expression level of each of the genes in the synovial tissue-derived cell or the meniscus-derived cell.

5. The composition for cartilage repair according to claim 4, wherein the three-dimensionally cultured product contains an umbilical cord tissue-derived cell cultured in the presence of a scaffold substrate.

6. The composition for cartilage repair according to claim 5, wherein expression levels of one or two kinds of Oct4 and Nanog genes in the umbilical cord tissue-derived cell are higher than an expression level of each of the genes in an umbilical cord tissue-derived cell cultured in the absence of the scaffold substrate.

7. The composition for cartilage repair according to claim 1, wherein a cartilage in the composition for cartilage repair is one or a plurality of kinds of cartilages selected from the group consisting of: a meniscus; an articular cartilage; an intervertebral disc; a costal cartilage; a tracheal cartilage; a bronchial cartilage; a nasal cartilage; a thyroid cartilage; a pubic symphysis; an articular disc; an epiphyseal plate; an auricular cartilage; a meatal cartilage; an epiglottic cartilage; a laryngeal cartilage; and an articular lip.

8. The composition for cartilage repair according to claim 1, wherein the umbilical cord tissue-derived cell includes an undifferentiated cell.

9. A production method for a composition for cartilage repair, comprising a step of three-dimensionally culturing an umbilical cord tissue-derived cell.

10. The production method according to claim 9, wherein the step of three-dimensionally culturing the umbilical cord tissue-derived cell includes a step of culturing the umbilical cord tissue-derived cell in the presence of a scaffold substrate.
